(19) United States
(12) Patent Application Publication
(10) Pub. No.: US 2022/0192539 A1
(43) Pub. Date: Jun. 23, 2022

(54) STIFF SHOULDER EVALUATION METHOD AND STIFF SHOULDER EVALUATION DEVICE

(71) Applicants: The University of Tokyo, Tokyo (JP); Murata Manufacturing Co., Ltd., Nagaokakyo-shi, Kyoto-fu (JP)

(72) Inventors: Takaei KIHARA, Nagaokakyo-shi (JP); Motoyasu NAKAO, Nagaokakyo-shi (JP); Ko MATSUDAIRA, Tokyo (JP); Junji KATSUHIRA, Tokyo (JP)

(21) Appl. No.: 17/654,084

(22) Filed: Mar. 9, 2022

Related U.S. Application Data
(63) Continuation of application No. PCT/JP2020/041660, filed on Nov. 9, 2020.

(30) Foreign Application Priority Data
Nov. 20, 2019 (JP) 2019-209319

(51) Int. Cl. A61B 5/11; A61B 5/00

(52) U.S. Cl. CPC A61B 5/1107; A61B 5/4519

(57) ABSTRACT

A stiff shoulder evaluation device includes a simultaneous contraction index detection unit and a stiff shoulder evaluation unit. The simultaneous contraction index detection unit detects simultaneous contraction indexes of skeletal muscles at a plurality of positions and in antagonistic relationship with each other. The stiff shoulder evaluation unit evaluates a state of stiff shoulder from the simultaneous contraction index.

### CROSS REFERENCE TO RELATED APPLICATION

This is a continuation of International Application No. PCT/JP2020/041660 filed on Nov. 9, 2020, which claims priority from Japanese Patent Application No. 2019-209319 filed on Nov. 20, 2019. The contents of these applications are incorporated herein by reference in their entireties.

### TECHNICAL FIELD

The present disclosure relates to a stiff shoulder evaluation technique for evaluating stiff shoulder.

### BACKGROUND ART

Patent Literature 1 recites a fatigue detection device using a neck band. The neck band includes a biological electrode and a fatigue determining unit.

The biological electrode acquires a biological signal from a neck of a user. The fatigue determining unit determines whether or not the user is fatigued on the basis of a myoelectric component obtained from the biological signal.

### CITATION LIST

### Patent Literature

[Patent Literature 1]
WO 2017/086073 A

### SUMMARY

### Technical Problem

The fatigue detection device recited in Patent Literature 1, however, may not be able to accurately evaluate a state of stiff shoulder.

Therefore, an object of the present disclosure is to provide a stiff shoulder evaluation technique that enables more accurate evaluation of a state of stiff shoulder.

### Solution to Problem

A stiff shoulder evaluation method according to the present disclosure detects simultaneous contraction of skeletal muscles at a plurality of positions and in antagonistic relationship with each other, and evaluates a state of stiff shoulder from a detection result of the simultaneous contraction.

While conventionally, it has been sensorily known that a state of a muscle around a neck affects stiff shoulder to some extent, the inventors have confirmed for the first time before others by experiments and the like that there is a correlation between a state of simultaneous contraction of skeletal muscles in antagonistic relationship with each other and a state (degree) of stiff shoulder. Therefore, this method enables a state of stiff shoulder to be evaluated by using a detection result of simultaneous contraction of skeletal muscles at a plurality of positions and in antagonistic relationship with each other.

### Advantageous Effects

According to the present disclosure, a state of stiff shoulder can be evaluated more accurately.

### BRIEF DESCRIPTION OF DRAWINGS

FIG. 1 is a functional block diagram illustrating a configuration of a stiff shoulder evaluation device according to a first embodiment.

FIG. 2 is a view illustrating a mounting position of a sensor.

FIG. 3 is a diagram for explaining a concept of calculating a simultaneous contraction index.

FIG. 4 is a graph illustrating a relationship between a simultaneous contraction index and a shoulder stiffness index.

FIG. 5 is a flowchart illustrating main processing of a stiff shoulder evaluation method according to the first embodiment.

FIG. 6 is a functional block diagram illustrating a configuration of a stiff shoulder evaluation device according to a second embodiment.

FIG. 7 is a flowchart illustrating main processing of a stiff shoulder evaluation method according to the second embodiment.

FIG. 8 is a flowchart illustrating main processing of a stiff shoulder evaluation method according to a third embodiment.

### DESCRIPTION OF EMBODIMENTS

### First Embodiment

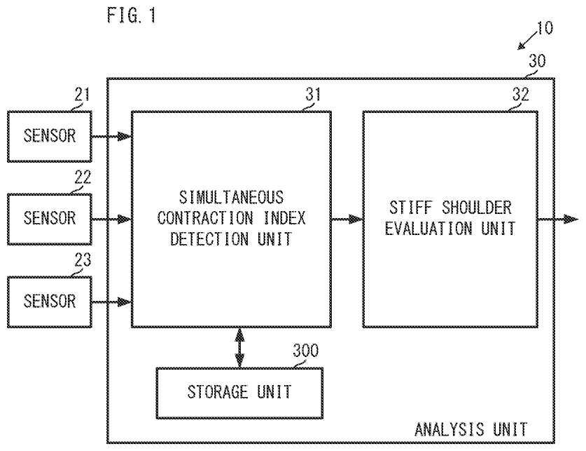

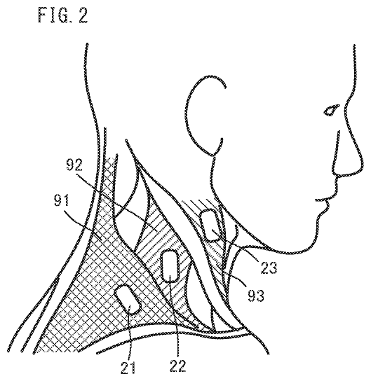

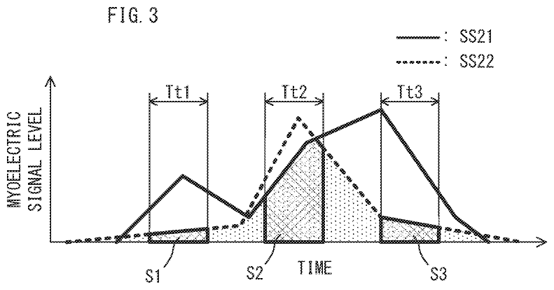

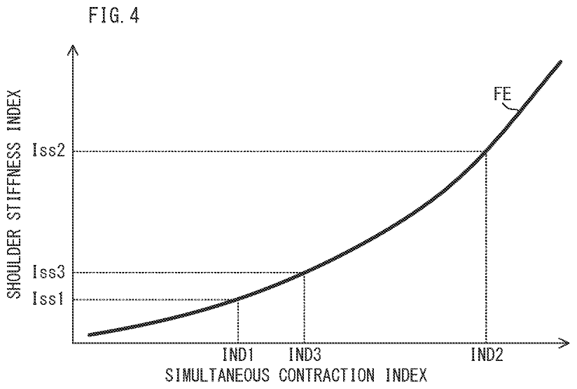

A stiff shoulder evaluation technique according to a first embodiment of the present disclosure will be described with reference to the drawings. FIG. 1 is a functional block diagram illustrating a configuration of a stiff shoulder evaluation device according to the first embodiment. FIG. 2 is a view illustrating a mounting position of a sensor. FIG. 3 is a diagram for explaining a concept of calculating a simultaneous contraction index. FIG. 4 is a graph illustrating a relationship between a simultaneous contraction index and a shoulder stiffness index.

As illustrated in FIG. 1, a stiff shoulder evaluation device 10 includes a sensor 21, a sensor 22, a sensor 23, and an analysis unit 30. The sensor 21, the sensor 22, the sensor 23, and the analysis unit 30 each have a configuration capable of data communication. The data communication may be wireless communication or wired communication.

The sensor 21, the sensor 22, and the sensor 23 have the same configuration. Each of the sensor 21, the sensor 22, and the sensor 23 is, for example, a biological signal sensor such as a myoelectric sensor, and includes a measurement electrode and a signal processing circuit which are not shown. The measurement electrode is disposed so as to be in contact with a subject, to acquire a biological signal (e.g., a myoelectric signal) from the subject. The signal processing circuit executes amplification of the biological signal, transmission of the biological signal to the analysis unit 30, and the like.

The sensor 21, the sensor 22, and the sensor 23 continuously acquire the biological signal, for example, at each measurement timing set at a predetermined time interval. Then, the sensor 21, the sensor 22, and the sensor 23 transmit the acquired biological signal at each transmission timing set at a predetermined time interval. The measurement timing and the transmission timing may correspond to each other on a one-to-one basis, and the number of transmission timings per unit time may be smaller than the number of measurement timings. The one-to-one correspondence eliminates the need for synchronization among the sensor 21, the sensor 22, and the sensor 23, and the number of communications between the plurality of sensors 21, 22, and 23 and the analysis unit 30 can be reduced by making the number of transmission timings per unit time be smaller than the number of measurement timings.

As illustrated in FIG. 2, the sensor 21 is installed so as to overlap a trapezius 91. The sensor 21 detects and outputs a biological signal whose level changes according to activity (muscle contraction or the like) of the trapezius 91. The sensor 22 is installed so as to overlap a scalenus 92. The sensor 22 detects and outputs a biological signal whose level changes according to activity (muscle contraction or the like) of the scalenus 92. The sensor 23 is installed so as to overlap a sternocleidomastoid muscle 93. The sensor 23 detects and outputs a biological signal whose level changes according to activity (muscle contraction or the like) of the sternocleidomastoid muscle 93.

Note that the present embodiment shows a mode in which the three sensors 21, 22, and 23 disposed on different muscles are used. However, two or more sensors disposed on different muscles are sufficient.

The present embodiment shows a mode in which the sensors are disposed on the trapezius 91, the scalenus 92, and the sternocleidomastoid muscle 93, respectively. However, it is also possible to dispose the sensor for other muscle in pair with and in antagonistic relationship with a skeletal muscle related to the shoulder and the neck.

The analysis unit 30 includes a simultaneous contraction index detection unit 31, a stiff shoulder evaluation unit 32, and a storage unit 300. The simultaneous contraction index detection unit 31 corresponds to a “simultaneous contraction detection unit” of the present disclosure. The simultaneous contraction index detection unit 31 and the stiff shoulder evaluation unit 32 are realized by, for example, an arithmetic processing device such as a CPU, an IC, and a program executed by the arithmetic processing device and the IC. Note that this program is stored in the storage unit 300, for example. Furthermore, this program may be stored in an external server or the like and acquired from the server.

The storage unit 300 is realized by a semiconductor storage medium, a magnetic storage medium, or the like.

Schematically, the simultaneous contraction index detection unit 31 detects a simultaneous contraction index IND using the biological signals from the sensor 21, the sensor 22, and the sensor 23, and outputs the simultaneous contraction index IND to the stiff shoulder evaluation unit 32. The stiff shoulder evaluation unit 32 evaluates a shoulder stiffness index Iss according to a state (degree) of stiff shoulder using the simultaneous contraction index IND.

More specifically, the simultaneous contraction index detection unit 31 sequentially acquires the biological signals from the sensor 21, the sensor 22, and the sensor 23, and stores and accumulates the biological signals in the storage unit 300. The simultaneous contraction index detection unit 31 stores in advance a sampling time length Tt for index detection.

The simultaneous contraction index detection unit 31 acquires biological signals of the plurality of sensors corresponding to the sampling time length Tt from the storage unit 300. The simultaneous contraction index detection unit 31 calculates an area S in which time waveforms of the acquired biological signals of the plurality of sensors overlap each other, and detects the simultaneous contraction index IND from the area S.

For example, in the example of FIG. 3, the simultaneous contraction index detection unit 31 sets sampling time lengths Tt1, Tt2, and Tt3 at different times corresponding to the sampling time length Tt. The sampling time lengths Tt1, Tt2, and Tt3 have the same length.

The simultaneous contraction index detection unit 31 acquires, from the storage unit 300, a biological signal SS21 (a biological signal corresponding to the activity of the trapezius 91) acquired by the sensor 21 and a biological signal SS22 (a biological signal corresponding to the activity of the scalenus 92) acquired by the sensor 22, which correspond to the sampling time length Tt1. From a level of the biological signal SS21 and a level of the biological signal SS22 at the same time (which may be substantially the same time), the simultaneous contraction index detection unit 31 detects an overlap (e.g., a level of the biological signal having the lower level) of the biological signals. The simultaneous contraction index detection unit 31 detects the overlap for the sampling time length Tt1.

The simultaneous contraction index detection unit 31 integrates the overlaps corresponding to the sampling time length Tt1 to calculate an area S1. A relationship between the area S and the simultaneous contraction index IND is stored in advance. Using this relationship, the simultaneous contraction index detection unit 31 detects a simultaneous contraction index IND1 from the area S1.

Similarly, the simultaneous contraction index detection unit 31 calculates an area S2 for the sampling time length Tt2 to detect a simultaneous contraction index IND2. In addition, the simultaneous contraction index detection unit 31 calculates an area S3 for the sampling time length Tt3 to detect a simultaneous contraction index IND3.

The simultaneous contraction index detection unit 31 outputs the simultaneous contraction indexes IND1, IND2, and IND3 to the stiff shoulder evaluation unit 32. The simultaneous contraction index detection unit 31 may store the simultaneous contraction indexes IND1, IND2, and IND3 and simultaneously output them to the stiff shoulder evaluation unit 32.

In the above example, the simultaneous contraction index detection unit 31 sets the sampling time length Tt at time intervals. The present disclosure is, however, not limited thereto, and for example, a plurality of sampling time lengths Tt may be continuous or partially overlapped. The set number of sampling time lengths Tt is not limited to three, and may be another number.

Furthermore, the above example shows a mode in which the biological signal SS21 of the sensor 21 and the biological signal SS22 of the sensor 22 are used. The combination of the biological signals is, however, not limited thereto, and as described above, biological signals acquired from a plurality of skeletal muscles in antagonistic relationship may be combined.

The stiff shoulder evaluation unit 32 stores in advance a relationship between the simultaneous contraction index IND and the shoulder stiffness index Iss such as an evaluation function FE illustrated in FIG. 4. The stiff shoulder evaluation unit 32 evaluates the shoulder stiffness index Iss using the simultaneous contraction index IND and the evaluation function FE. For example, in the example of FIG. 4, the stiff shoulder evaluation unit 32 evaluates a shoulder stiffness index Iss1 from the simultaneous contraction index IND1, evaluates a shoulder stiffness index Iss2 from the simultaneous contraction index IND2, and evaluates a shoulder stiffness index Iss3 from the simultaneous contraction index IND3 using the evaluation function FE.

Through various experiments, the inventors have found for the first time that there is a correlation between a degree of simultaneous contraction of skeletal muscles in the vicinity of a shoulder and a neck and in antagonistic relationship and a state (degree) of stiff shoulder. Specifically, as shown in FIG. 4, it has been found that when the degree of simultaneous contraction increases, the state of stiff shoulder deteriorates, in other words, the degree of stiff shoulder increases.

The degree of simultaneous contraction corresponds to a degree of simultaneous contraction of a plurality of skeletal muscles, and has a correlation (e.g., a proportional relationship) with the above-described area S. Therefore, the degree of simultaneous contraction can be expressed by the simultaneous contraction index IND described above.

The shoulder stiffness index Iss is set such that the degree of stiff shoulder increases as the value increases. Furthermore, the evaluation function FE is set according to the correlation between the degree of simultaneous contraction (simultaneous contraction index IND) and the degree of stiff shoulder (shoulder stiffness index Iss) on the basis of the above-described experiment and the like.

By using this relationship and setting, the stiff shoulder evaluation unit 32 can more accurately evaluate the degree of stiff shoulder using the shoulder stiffness index Iss.

Note that without using the evaluation function FE, the stiff shoulder evaluation unit 32 may set and store in advance a relationship table between the simultaneous contraction index IND and the shoulder stiffness index Iss, and may evaluate the shoulder stiffness index Iss with reference to the relationship table.

As described above, use of the configuration of the present embodiment enables the stiff shoulder evaluation device 10 to more accurately evaluate stiff shoulder. In particular, the stiff shoulder evaluation device 10 can more accurately evaluate stiff shoulder caused by a so-called straight neck.

In the above description, the stiff shoulder evaluation device 10 is configured including the sensor 21, the sensor 22, and the sensor 23. However, the analysis unit 30 described above can be used as the stiff shoulder evaluation device 10 as long as a means is separately provided for acquiring the biological signals measured from skeletal muscles in the vicinity of the shoulder and the neck and in antagonistic relationship.

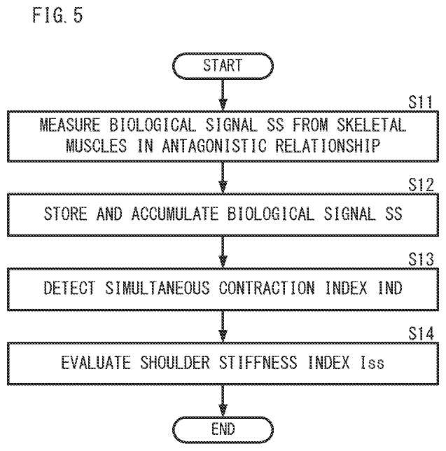

Although the above description has shown a mode in which each processing related to the stiff shoulder evaluation method according to the first embodiment is executed by an individual functional unit, each processing may be realized by the sensor and a program or the like executed by the arithmetic processing device. In this case, the stiff shoulder evaluation method illustrated in FIG. 5 may be used. FIG. 5 is a flowchart illustrating main processing of the stiff shoulder evaluation method according to the first embodiment. Note that specific contents of each processing have been described above, and description of specific processing will be omitted except for a part requiring additional description.

First, the sensors 21, 22, and 23 measure a biological signal SS of skeletal muscles in the vicinity of the shoulder and the neck and in antagonistic relationship (S 11). The arithmetic processing device stores and accumulates the biological signal SS (S 12).

The arithmetic processing device detects the simultaneous contraction index IND from the accumulated biological signal SS (S 13). The arithmetic processing device evaluates the shoulder stiffness index Iss from the simultaneous contraction index IND (S 14).

### Second Embodiment

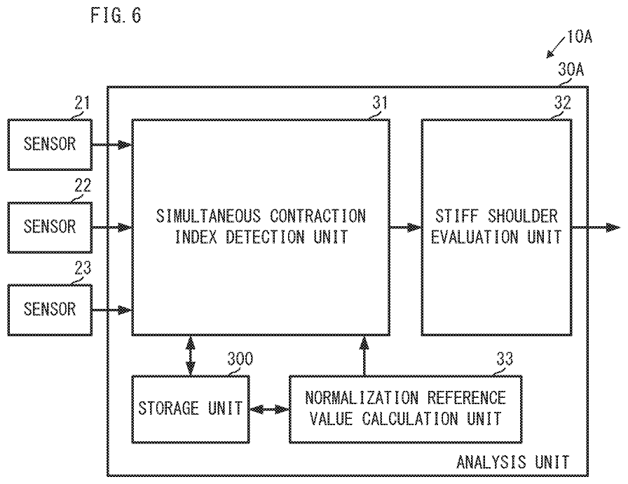

A stiff shoulder evaluation technique according to a second embodiment of the present disclosure will be described with reference to the drawings. FIG. 6 is a functional block diagram illustrating a configuration of a stiff shoulder evaluation device according to the second embodiment.

As illustrated in FIG. 6, a stiff shoulder evaluation device 10A according to the second embodiment is different from the stiff shoulder evaluation device 10 according to the first embodiment in a configuration of an analysis unit 30A. The other configurations of the stiff shoulder evaluation device 10A are similar to those of the stiff shoulder evaluation device 10, and description of the similar parts will be omitted.

The analysis unit 30A is different from the analysis unit 30 according to the first embodiment in that a normalization reference value calculation unit 33 is included, and in various kinds of processing related thereto. The other configurations and processing of the analysis unit 30A are similar to those of the analysis unit 30, and description of the similar parts will be omitted.

The normalization reference value calculation unit 33 calculates a normalization reference value of a biological signal when calculating a simultaneous contraction index IND. More specifically, the normalization reference value is calculated by performing the following processing.

A storage unit 300 stores a biological signal for performing normalization. Acquisition of a biological signal (normalization biological signal) for performing normalization is performed separately from acquisition of the biological signal for evaluating stiff shoulder. The normalization biological signal is measured while applying an excessive load to, for example, a trapezius 91 a scalenus 92, and a sternocleidomastoid muscle 93 on which a sensor 21, a sensor 22, and a sensor 23 are disposed. More specifically, the normalization biological signal is a biological signal measured at timing when a load is applied to a subject until the contractions of the trapezius 91, the scalenus 92, and the sternocleidomastoid muscle 93 reach their maximums in the subject. The normalization reference value calculation unit 33 calculates a level of the normalization biological signal as a normalization reference value. At this time, the normalization reference value calculation unit 33 calculates the normalization reference value for each skeletal muscle, for example, for each of the trapezius 91, the scalenus 92, and the sternocleidomastoid muscle 93 in this case.

The normalization reference value calculation unit 33 outputs the normalization reference value for each skeletal muscle to a simultaneous contraction index detection unit 31.

The simultaneous contraction index detection unit 31 normalizes the biological signal with the normalization reference value for each skeletal muscle. For example, the simultaneous contraction index detection unit 31 normalizes the biological signal by dividing the level of the biological signal by the normalization reference value.

The simultaneous contraction index detection unit 31 detects the simultaneous contraction index IND using a normalized biological signal, similarly to the first embodiment described above. Then, a stiff shoulder evaluation unit 32 evaluates a shoulder stiffness index Iss from the simultaneous contraction index IND based on the normalized biological signal.

By using such a configuration and processing, the simultaneous contraction index detection unit 31 can detect the simultaneous contraction index IND according to a subject and a state of the subject. As a result, the stiff shoulder evaluation unit 32 can evaluate the shoulder stiffness index Iss according to the subject and the state of the subject. Therefore, the stiff shoulder evaluation device 10A can more accurately evaluate a state of stiff shoulder according to the subject and the state of the subject.

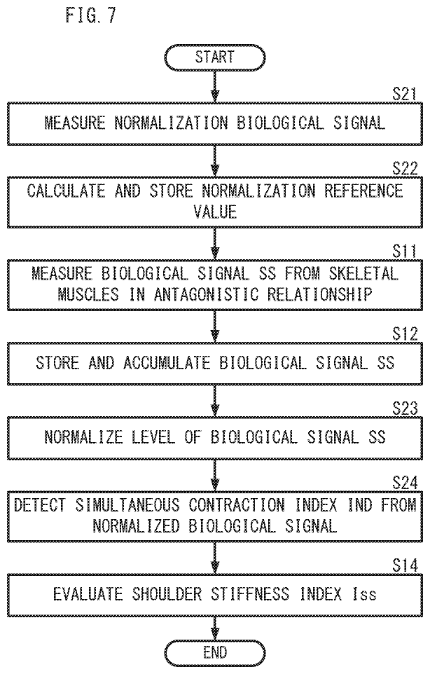

Although the above description has shown a mode in which each processing related to the stiff shoulder evaluation method according to the second embodiment is executed by the individual functional unit, these processing may be realized by the sensor and a program or the like executed by an arithmetic processing device. In this case, the stiff shoulder evaluation method illustrated in FIG. 7 may be used. FIG. 7 is a flowchart illustrating main processing of the stiff shoulder evaluation method according to the second embodiment. Note that specific contents of each processing have been described above, and description of specific processing will be omitted except for a part requiring additional description.

The sensors 21, 22, and 23 measure normalization biological signals for skeletal muscles in the vicinity of the shoulder and the neck and in antagonistic relationship (S 21). The arithmetic processing device calculates and stores a normalization reference value from the normalization biological signals (S 22).

The sensors 21, 22, and 23 measure a biological signal SS of the skeletal muscles in the vicinity of the shoulder and the neck and in antagonistic relationship (S 11). The arithmetic processing device stores and accumulates the biological signal SS (S 12).

The arithmetic processing device normalizes a level of the biological signal SS using the normalization reference value (S 23).

The arithmetic processing device detects the simultaneous contraction index IND from the normalized biological signal (S 24). The arithmetic processing device evaluates the shoulder stiffness index Iss from the simultaneous contraction index IND (S 14).

### Third Embodiment

A stiff shoulder evaluation technique according to a third embodiment of the present disclosure will be described with reference to the drawings. A configuration of a stiff shoulder evaluation device of the third embodiment is similar to that of the stiff shoulder evaluation device described in each of the above-described embodiments, and description of the similar parts will be omitted.

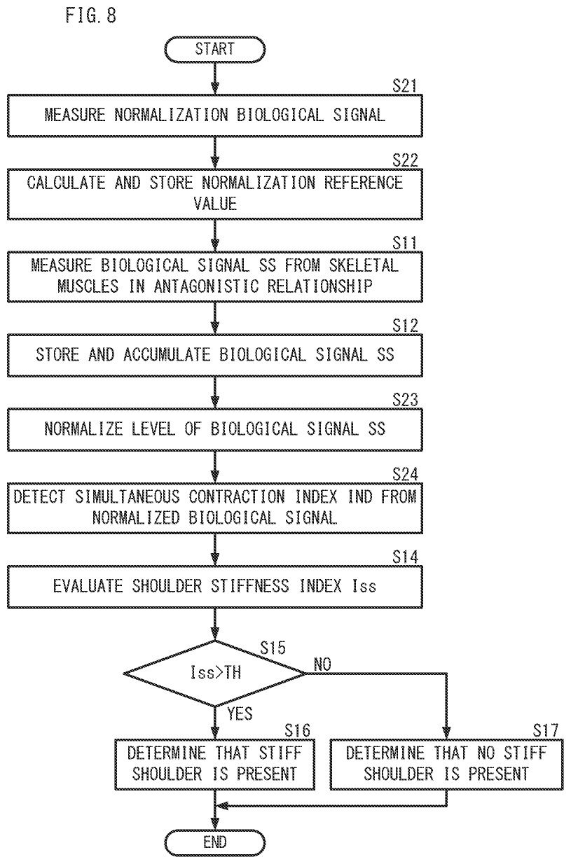

FIG. 8 is a flowchart illustrating main processing of the stiff shoulder evaluation method according to the third embodiment. As illustrated in FIG. 8, the stiff shoulder evaluation method according to the third embodiment is different from the stiff shoulder evaluation method according to the second embodiment in that processing of determining presence or absence of stiff shoulder is added. The other processing of the stiff shoulder evaluation method according to the third embodiment is similar to that of the stiff shoulder evaluation method according to the second embodiment, and description of the similar parts will be omitted.

An arithmetic processing device stores in advance a threshold value TH for determining the presence or absence of stiff shoulder. The threshold value TH can be set on the basis of, for example, past experimental results or the like.

When a shoulder stiffness index Iss is larger than the threshold value TH (S 15: YES), the arithmetic processing device determines that “the stiff shoulder is present” (S 16). When the shoulder stiffness index Iss is equal to or less than the threshold value TH (S 15: NO), the arithmetic processing device determines that “no stiff shoulder is present” (S 17).

Note that although in each of the embodiments described above, the stiff shoulder evaluation device and the stiff shoulder evaluation method have the configuration and the method for performing evaluation of a state of stiff shoulder and determination of presence or absence of stiff shoulder, the stiff shoulder evaluation device and the stiff shoulder evaluation method may have a function of notifying these evaluation results or determination results to the outside. For example, the stiff shoulder evaluation device may notify the evaluation result or the determination result by an image, voice, or the like, or may notify these results to an app or the like of a smartphone owned by a subject.

Furthermore, although each of the above-described embodiments has shown a mode in which a myoelectric signal is used as a biological signal, other biological signals can be applied as long as the signals change in state according to activity of a skeletal muscle.

In addition, the configurations and processing of the above-described embodiments can be appropriately combined to exhibit functions and effects according to the respective combinations.

### REFERENCE SIGNS LIST

10, 10A stiff shoulder evaluation device
21, 22, 23 sensor
30, 30A analysis unit
31 simultaneous contraction index detection unit
32 stiff shoulder evaluation unit
33 normalization reference value calculation unit
91 trapezius
92 scalenus
93 sternocleidomastoid muscle
300 storage unit

1. A stiff shoulder evaluation method comprising:
detecting simultaneous contraction of at least two skeletal muscles, the at least two skeletal muscles being in an antagonistic relationship with each other; and
evaluating a state of stiff shoulder based on the detected simultaneous contraction.

2. The stiff shoulder evaluation method according to claim 1, further comprising:
measuring biological signals from the at least two skeletal muscles,
wherein the biological signals measured from the at least two skeletal muscles are used to detect the simultaneous contraction.

3. The stiff shoulder evaluation method according to claim 2, wherein overlapping time waveforms of the biological signals measured from the at least two skeletal muscles are used to detect the simultaneous contraction.

4. The stiff shoulder evaluation method according to claim 3, further comprising:
normalizing the measured biological signals,
wherein the normalized biological signals are used to detect the simultaneous contraction.

5. The stiff shoulder evaluation method according to claim 1, wherein the at least two skeletal muscles include at least two of a trapezius, a scalenus, and a sternocleidomastoid muscle.

6. A stiff shoulder evaluation device comprising:
at least one processor and memory, the at least one processor being configured to:
detect simultaneous contraction of at least two skeletal muscles, the at least two skeletal muscles being in an antagonistic relationship with each other; and
evaluate a state of stiff shoulder based on the detected simultaneous contraction.

7. The stiff shoulder evaluation device according to claim 6, further comprising:
a plurality of sensors that are respectively disposed on a corresponding one of the at least two skeletal muscles, the plurality of sensors each being configured to measure a biological signal from the corresponding skeletal muscle and to output the measured biological signal to the at least one processor or memory,
wherein the at least one processor is configured to detect the simultaneous contraction using the measured biological signals.

8. The stiff shoulder evaluation device according to claim 7, wherein the at least one processor is configured to detect the simultaneous contraction by using overlapping time waveforms of the biological signals measured from the at least two skeletal muscles.

9. The stiff shoulder evaluation device according to claim 8, wherein the at least one processor is further configured to determine normalization reference values of the biological signals and to normalize the biological signals based on the normalization reference values, and
wherein the at least one processor is configured to detect the simultaneous contraction using the normalized biological signals.

10. The stiff shoulder evaluation device according to claim 6, wherein the at least two skeletal muscles include at least two of a trapezius, a scalenus, and a sternocleidomastoid muscle.
